SEQUENCE LISTING (10 sequences)

SEQ ID NO: 1 - RNA, 21 nt, Artificial Sequence
  Description of Artificial Sequence A forward designed primer which is used for modification and amplification of target nucleic acid
uccuccugagcgcaaguacuc

SEQ ID NO: 2 - DNA, 21 bp, Artificial Sequence
  Description of Artificial Sequence A forward designed primer which is used for modification and amplification of target nucleic acid
cctgcttgctgatccacatct

SEQ ID NO: 3 - DNA, 21 bp, Artificial Sequence
  Description of Artificial Sequence Designed chip probe which is use for hybridization of the partially double-stranded target nucleic acid
tcctcctgagcgcaagtactc

SEQ ID NO: 4 - RNA, 18 nt, Artificial Sequence
  Description of Artificial Sequence A forward designed primer which is used for modification and amplification of target nucleic acid
uggucguauugggcgccu

SEQ ID NO: 5 - DNA, 22 bp, Artificial Sequence
  Description of Artificial Sequence A reverse designed primer which is used for modification and amplification of target nucleic acid
accctgttgctgtagccaaatt

SEQ ID NO: 6 - DNA, 29 bp, Artificial Sequence
  Description of Artificial Sequence A reverse designed primer which is used for modification and amplification of target nucleic acid
catattggaacatgtaaaccatgtagttg

SEQ ID NO: 7 - DNA, 21 bp, Artificial Sequence
  Description of Artificial Sequence A reverse designed primer which is used for modification and amplification of target nucleic acid
ttgattttggagggatctcgc

SEQ ID NO: 8 - DNA, 21 bp, Artificial Sequence
  Description of Artificial Sequence A reverse designed primer which is used for modification and amplification of target nucleic acid
gctaagcagttggtggtgcag

SEQ ID NO: 9 - DNA, 18 bp, Artificial Sequence
  Description of Artificial Sequence Designed chip probe which is used for hybridization of the partially double-stranded target nucleic acid
tggtcgtattgggcgcct

SEQ ID NO: 10 - DNA, 23 bp, Artificial Sequence
  Description of Artificial Sequence Designed chip probe  which is used for hybridization of the partially double-stranded target nucleic acid
cctctgacttcaacagcgacact

(19) United States
(12) Patent Application Publication
(10) Pub. No.: US 2005/0095606 A1
(43) Pub. Date: May 5, 2005

(54) Partially double-stranded nucleic acids, methods of making, and use thereof

(76) Inventors: Glenn Hoke, Mt. Airy, MD (US); John Hartwell, Silver Spring, MD (US); Adam Steel, Silver Spring, MD (US)

Correspondence Address: BLANK ROME LLP, 600 NEW HAMPSHIRE AVENUE, N.W., WASHINGTON, DC, 20037, US

(21) Appl. No.: 10/757,468

(22) Filed: Jan. 15, 2004

Related U.S. Application Data
(63) Continuation of application No. 09/984,517, filed on Oct. 30, 2001, now abandoned.

(51) Int. Cl. C12Q 1/68; C12P 19/34

(52) U.S. Cl. 435/6; 435/91.2

(57) ABSTRACT

A process is disclosed for generating at least one partially double-stranded target nucleic acid, which contains at least one single-stranded region at a terminal end. The process comprises the steps of (a) providing at least one primer, P1, containing at least one labile nucleotide; (b) combining at least one target nucleic acid sequence with P1 to generate a double-stranded polynucleotide containing at least one labile nucleotide; (c) exposing the double-stranded polynucleotide to conditions that promote single-strand cleavage of the polynucleotide at the site of the at least one labile nucleotide of primer P1; and (d) exposing the cleaved polynucleotide to conditions that promote the dissociation of the cleaved portions of primer P1 from a terminal end. The labile nucleotide may be dUTP, wherein the single-stranded cleavage of the polynucleotide at the site of the labile nucleotide occurs by treatment with uracil N-glycosylase.

### FIELD OF THE INVENTION

The present invention provides methods for generating partially double-stranded nucleic acid molecules that contain at least one terminal single-stranded region available for binding to a complementary nucleic acid. Such molecules are useful for a wide variety of applications, including gene expression analysis, and are particularly useful as targets in DNA microarray analysis and related studies. Because these molecules may be generated without performing a strand separation step, they provide a substantial advantage over traditionally amplified target molecules. In combination with a bipartite primer sequence, the present method provides a universal DNA array index system that can detect or determine the amount of any polynucleotide target.

### BACKGROUND OF THE INVENTION

Tens of thousands of genes, and potential genes, have been identified in the human genome alone. Many times that number are known and suspected in medically and economically critical organisms ranging from viruses, bacteria, and fungi to crop and forestry plants. Irrespective of the organism, a complex web of signals directs each gene to remain quiescent, or to initiate some level of activity. Activity, or “gene expression,” is characterized by the production of a single-strand molecule of RNA. Many of these nucleic acids are subsequently processed into messenger RNA (mRNA), intermediate molecules which are translated into the proteins encoded by their respective genes. Consequently, an assessment of the level of an mRNA or equivalent transcript in a virus or non-eukaryotic organism provides insight into expression of both the gene and its encoded protein.

Such analyses provide powerful Insights into the cellular processes that occur in normal and disease states. For example, the expression profile of one or more genes in normal cells can be compared to that of cancerous, infected, or otherwise diseased cells to provide information regarding the identity of genes affected in the disease state. This information can provide insights that are useful in developing treatments for the disease, or in understanding the pathology of the disease. For example, the increased expression of a gene in tumor cells may point to an underlying cause for the malignancy, whereas increased expression of a gene in response to infection may be indicative of its role in combating that disease.

The detection of the presence or absence of gene activity and/or the determination of the amount of gene activity was once dependent on difficult and labor intensive procedures such as Northern hybridization, and later quantitative PCR (polymerase chain reactions). But new techniques are available to dramatically increase the number of mRNAs that can be assessed, and these methods have been adapted to efficiently assay a wide range of biological molecules. In particular, scientists have come to rely on microfabricated arrays (“microarrays”) of drugs, nucleic acids, peptides, etc., each member of the array having a distinct chemical, nucleotide, or amino acid sequence for potential binding or detection. Microarrays comprising large numbers of oligonucleotide probes are commonly called “DNA chips,” and offer great promise for a wide variety of applications. DNA chips have proven useful in generating gene expression profiles for many different biological materials, such as cells, tissues, viruses, fungi, parasites, microorganisms,. etc. Cell populations from these sources may be made up of cells, which differ in cell type, tissue type, physiological state, disease state, or developmental stage.

In addition to differentiating normal and diseased cell populations, DNA chips are useful in comparing expression profiles for drug-treated and untreated cells. Exemplary applications include evaluating the effectiveness of a course of treatment in a patient, detecting a history of illicit drug use, or for in vitro screening of potential drug candidates for treating a disease or physiological condition.

A DNA chip is basically a device for hybridizing a single strand region of a nucleic acid probe to a complementary single strand region of a nucleic acid target, and then detecting the bound product. Typically, this involves a microarray containing many thousands of unique DNA probes fixed to a solid support, or chip. This support may be fabricated from a wide variety of materials, for example, plastics, glass, or silicon derivatives.

In practice, a mixture containing nucleic acids derived from the cells or tissues of interest is applied to the chip such that target nucleic acid molecules within the mixture specifically hybridize with the bound probes and are retained on the chip. Other nucleic acids in the mixture are simply washed away. Once separated from the mixture, bound target nucleic acids are detected or quantified using standard methods. Most commonly, these methods rely on a detectable label contained within the target.

One method of marking the target nucleic acids with a detectable label employs the polymerase chain reaction (PCR) to incorporate labeled oligonucleotide primers, or individual labeled nucleotides, during nucleic acid synthesis. PCR also offers the advantage of exponentially increasing the amount of a specific nucleic acid target, thereby increasing the likelihood of detecting rare nucleic acid sequences. However, conventional PCR results in double-stranded nucleic acid products, whereas DNA chip technology is based on the binding of complementary single-stranded regions on the probe and target. Consequently, the double-stranded products of conventional PCR require further processing to generate appropriately single-strand molecules. Such processing usually involves denaturing the entire PCR product into two complete, entirely complementary single strands using heat, alkali, or other chaotropic agents. This strand separation step can be problematic and inefficient because the complementary strands tend to rapidly rehybridize to each other before attaching to the microarray.

In view of the great importance of DNA chip technology, a need exists for improved methods for generating PCR products that can be readily hybridized to a DNA microarray without requiring a strand separation step.

### BRIEF DESCRIPTION OF THE FIGURES

FIG. 1 illustrates how the deoxyuracil-containing primer could be used to generate fluorescently labeled, sequence-specific amplicons through RT-PCR (reverse transcriptase-polymerase chain reaction) amplification.

FIG. 2 illustrates how UNG-treated amplicons can be captured by a probe attached to a microarray surface.

FIG. 3 illustrates how a Flow-Thru Chip™ can capture an UNG-treated amplicon.

FIG. 4 illustrates how a universal index chip can be used to detect different target nucleic acids.

FIG. 5 details the structure and composition of a representative primers and probes, including degradeable deoxyuracil-containing oligonucleotide primers.

FIG. 6 illustrates the production of a single-stranded overhang region in PCR amplicon by degradation of a deoxyuracil-containing primer by UNG and base via a gel shift assay.

FIG. 7 illustrates multiplex PCR preparation of multiple single-stranded overhang containing amplicons of various length by degradation of a deoxyuracil-containing primer by UNG and base via a sizing gel.

FIG. 8 depicts the level of expression of twelve target nucleic acids from two different cell populations, tamoxifen-treated cells and estradiol-treated cells, compared to a control cell.

### SUMMARY OF THE INVENTION

The present invention relates to: 1) labile (modifiable) oligonucleotide primers for DNA polymerases; 2) differentially labile polynucleotides generated from such primers; and 3) partially double-stranded polynucleotides derived from the differentially labile polynucleotides. One or more labile moieties provides the lability of the above sequences, such as 5-hydroxy-2′-deoxycytidine, ribonucleic acids, or deoxyuracil, incorporated in lieu of standard deoxynucleotides (DNA bases), at some or all positions in an oligonucleotide primer.

The invention further comprises methods for the synthesis and use of the above polynucleotides. In an exemplary embodiment, an oligonucleotide primer having one or more deoxyuracil bases is used in a polymerase chain reaction (“PCR”), or other polymerase reaction, to synthesize a differentially labile double-stranded molecule. Enzymatic digestion of the deoxyuracil aids in generating single-strand breaks in the end of the double-stranded molecule, to thereby fragment the labile primer. These fragments have a lower melting temperature than the remainder of the molecule and are easily disassociated from it. The resulting molecule is, thus, partially double-stranded but has a single-stranded region generally corresponding to the labile primer. The single-stranded region is then available to hybridize with a complementary target sequence, and may be used to bind, detect, or quantify a target molecule, e.g., in a nucleic acid microarray.

The invention further relates to a universal index system comprising a primer containing two distinct regions, a bipartite primer. In one embodiment, the 5′ region of the primer comprises a standard sequence, which may be a random sequence. This portion of the primer contains at least one labile nucleotide. The 3′ portion of the bipartite primer comprises any sequence specific for a target nucleic acid of interest, for example, an mRNA or the nucleic acid of a pathogen. Amplification of the target using the bipartite primer, followed by cleavage of the labile nucleotides, provides a partially double-stranded nucleic acid having a single-stranded region corresponding to the complement of the standard sequence. This molecule can then be hybridized to a probe comprising the standard sequence.

Consequently, a universal test kit or DNA array comprising the standard sequence probe affixed to a solid support may be used to detect or determine any target, merely by varying the target-specific 3′ end of the bipartite primer. Moreover, the standardization afforded by the universal system allows for accurate comparison of target levels irrespective of the time or place of the assay.

### DETAILED DESCRIPTION OF THE INVENTION

In one basic embodiment, modified, partially double-stranded polynucleotides containing at least one single-stranded region at a terminal end are prepared by:
1. providing at least one primer, P1, containing at least one labile nucleotide;
2. combining at least one target nucleic acid sequence with P1 to generate a double-stranded polynucleotide containing at least one labile nucleotide;
3. exposing the double-stranded polynucleotide to conditions that promote single-strand cleavage of the polynucleotide at the site of the at least one labile nucleotide of primer P1; and
4. exposing the cleaved polynucleotide to conditions that promote the dissociation of the cleaved portions of primer P1 from a terminal end.

In some embodiments, at least one primer, P1, may be used in conjunction with at least one primer, P1, to amplify one or more target nucleic acid sequences. P1 primers optionally contain at least one labile nucleotide. P1, P1, or both may contain a detectable label.

At least one primer used to make the partially double-stranded target nucleic acid may contain a detectable label. A detectable label may also be incorporated elsewhere into the partially double-stranded target during, for example, the amplification reaction. The detectable label may be a radioisotope, a chromophore, a fluorophore, an enzyme, a reactive group, or a double-stranded DNA selective reagent.

The target nucleic acid to be amplified may be any nucleic acid, e.g., RNA, including mRNA, genomic DNA, cDNA, plasmid and recombinant DNA and amplified DNA. In some embodiments, a multiplicity of target nucleic acids is amplified in a reaction. The number of different target nucleic acids amplified may range from 2-200, 50-100, or 200-1000 or more, including any range of integers subsumed within these ranges. For example, a multiplicity of primers P1, having different base sequences and/or a multiplicity of primers P1 having different base sequences may be used to amplify a multiplicity of different target nucleic acids in a single reaction. In some embodiments, multiple different target nucleic acids may bind to the same primer P1, and/or primer P1 to generate a multiplicity of different amplicons. This may be the result of reduced hybridization stringency of a primer, or of differences in the target sequences outside of the region bound by a primer.

In one embodiment, the labile nucleotide is deoxyuridine triphosphate (“ddUTP”), which may be cleaved by treatment with uracil N-glycosylase (“UNG”).

In another embodiment the labile nucleotide is 5-hydroxy-2′-deoxycytidine triphosphate or 5-hydroxy-2′ deoxyuridine triphosphate, which may be cleaved by treatment with E. coli exonuclease III or with formamidopyrimidine DNA N-glycosylase.

In another embodiment, the labile nucleotide is a photolabile base and is cleaved by treatment with a particular wavelength of light.

In yet another embodiment, the labile nucleotide is a ribonucleotide and is cleaved by treatment with Rnase H.

In some embodiments, the labile nucleotide is a chemically linked base and is cleaved by treatment with a chemical that only cleaves at the site of the labile nucleotide.

The invention further encompasses a process for generating partially double stranded target nucleic acids containing at least one single-stranded region at a terminal end of the molecule. One embodiment of this process comprises:
1. preparing a first primer, P1, comprising at least one labile nucleotide, wherein P1, optionally contains a detectable label;
2. preparing a second primer, P1, wherein P1 may optionally contain at least one labile nucleotide and/or a detectable label;
3. amplifying the target nucleic acid with P1, and P1 generating a double stranded amplicon;
4. exposing the amplified target nucleic acid to conditions that promote cleavage of at least one labile nucleotide to generate at least one partially double-stranded target nucleic acid containing at least one single-stranded region at a terminal end.

According to another aspect of the invention, there is provided a method for detecting the presence or determining the amount of a partially double-stranded target nucleic acid prepared according to the process just described comprising hybridizing the partially double-stranded target to a set of nucleic acid probes. This set of nucleic acid probes may be attached to a solid support. In one embodiment, the nucleic acid probes comprise a nucleic acid microarray.

According to another aspect of the invention, there is provided a method for detecting the presence or determining the amount of at least one target nucleic acid sequence in a first sample of biological material relative to the same target nucleic acid sequence(s) in a second sample of biological material comprising:
1. preparing at least one partially double-stranded polynucleotide containing a target nucleic acid sequence and at least one single-stranded region at a terminal end for each target nucleic acid sequence from the first sample of biological material and from the second sample of biological material according to the process described above;
2. hybridizing the partially double-stranded polynucleotide from the first sample to a first set of nucleic acid probes and hybridizing the partially double-stranded polynucleotides from the second sample to a second set of nucleic acid probes;
3. detecting or determining the presence or amount of the partially double-stranded polynucleotide(s) from the first sample of biological material relative to the partially double-stranded polynucleotide(s) from the second sample of biological material.

In one embodiment, the first sample of biological material comprises one or more cells, a cell lysate, or subcellular fraction, and the second sample of biological material also comprises one or more cells, a cell lysate, or subcellular fraction, wherein the first and second sample may differ in, e.g., cell type, tissue type, physiological state, disease state, radiological, or biological treatment, or developmental stage. For example, the first sample may be chosen from a cancerous cell population of a particular cell type, and the second sample may be a reference cell chosen from the same cell type as the cancerous cell population. This reference cell may be a normal cell type, a cell chosen from a particular stage of cancer, or any cell type that can serve as a reference for comparison for the first sample.

In another embodiment, the primers used to amplify the partially double-stranded target nucleic acid from the first sample may be the same or different from the primers used to amplify the partially double-stranded target nucleic acid from the second sample. If the primers are different, they are still designed to amplify the same target nucleic acid.

In another embodiment, the second primer, P1, used to amplify the target nucleic acid may be labeled. When amplifying target nucleic acids from a first sample and a second sample, the label in the P1, which is used to amplify the target nucleic acid of the first sample, may be different from the label used in the other primer P1, which is used to amplify the target nucleic acid of the second sample. This would also allow one to determine the amount of the target nucleic acid from the first sample relative to the amount of the target nucleic acid from the second sample on the same solid support by comparing the signal of the first label relative to the second label.

In another embodiment, the first set of nucleic acid probes may be composed of the same nucleic acid probes as the second set. Both sets of nucleic acid probes may be attached to a solid support and may comprise a DNA microarray. The solid support may be chosen from capillary tubes, beads, slides, sheets, pins, microtiter plates, silicon, porous silicon porous metal oxide, plastic, polycarbonate, polystyrene, cellulose, glass, TEFLON(®), polystyrene divinyl benzene, aluminum, steel, iron, copper, nickel, silver, and gold.

According to another aspect of the invention, there is provided a double-stranded polynucleotide comprising three regions, wherein:
1. the first region comprises a single-stranded region of at least 8 nucleotides, wherein the single-stranded region is generated by:
   a) preparing a first primer, P1, comprising at least one labile (modifiable) nucleotide, wherein the first primer optionally contains a detectable label;
   b) preparing a second primer, P1, comprising a sequence that is specific for a target nucleic acid sequence and optionally contains a detectable label and/or at least one labile nucleotide;
   c) amplifying the target nucleic acid sequence with P1, and P1 generating a double stranded amplicon containing at least one labile nucleotide;
   d) exposing the double-stranded amplicon to conditions that promote single-strand cleavage of the amplicon at the site of at least one labile nucleotide; and
   e) exposing the cleaved amplicon to conditions which promote the dissociation of the cleaved portions from the first region; and
2. the second region comprises the sequence of the double-stranded amplicon between the first region and a third region; and
3. the third region comprises the sequence of the double-stranded amplicon comprising P1 and the complementary sequence to P1.

In one embodiment, P1 contains a detectable label, which is subsequently incorporated into the third region. In another embodiment a label is incorporated into P1 3′ to any labile nucleotide. In a third embodiment, a detectable label is incorporated into the second region.

In another embodiment, the single-stranded region may be 10-50 nucleotides or more in length, or any integer value subsumed within that range.

In another aspect of the invention, there is provided a method for generating a partially double-stranded polynucleotide containing at least one single-stranded index sequence at a terminal end comprising:

preparing a first primer, Pa, comprising two regions:

a) a first region comprising a first index sequence containing at least one labile nucleotide wherein, the first region is not complementary to a target nucleic acid sequence; and
b) a second region that is specific for the target nucleic acid sequence;
   preparing a second primer, Pb, comprising a sequence that is specific for the target nucleic acid sequence and optionally contains a detectable label and/or a second index sequence, wherein the second index sequence may be the same or different from the first index sequence of the first region of the first primer;
   amplifying the target nucleic acid sequence with Pa and Pb generating a double-stranded amplicon;
   exposing the amplicon to conditions that promote single-stranded cleavage of the amplicon at the site of the labile nucleotide(s);
   exposing the cleaved amplicon to conditions that promote the dissociation of the cleaved portions of the index region(s) of the primer(s) containing the labile nucleotide to generate a single-stranded region at the terminal end.

In yet another embodiment, the double-stranded amplicon is labeled. The primers may contain the label, label may be added during amplification, or the label may be added after cleavage of the double-stranded amplicon. If the double-stranded amplicon contains an index sequence at each end, a label may be added by hybridizing a nucleic acid complementary to the single-stranded index sequence, wherein the complementary nucleic acid contains a label. The complementary index sequence may also be enzymatically extended to incorporate at least one detectable label. The following may be used to label the target nucleic acid: a radioisotope, a chromophore, a fluorophore, an enzyme, an antigen, a reactive group, or a double-stranded DNA selective reagent.

Another aspect of the invention provides a method for determining the presence of a target nucleic acid in a first sample of biological material relative to a second sample of biological material comprising:
1. preparing a partially double-stranded target nucleic acid containing at least one single-stranded index sequence at a terminal end from the first sample of biological material and preparing a partially double-stranded target nucleic acid containing at least one single-stranded index sequence at a terminal end from the second sample of biological material according to the method just described;
2. hybridizing the single-stranded index sequence of the target nucleic acid from the first sample of biological material to a complementary single-stranded index sequence bound to a first solid support, wherein the solid support comprises at least one single-stranded index region;
3. hybridizing the single-stranded index sequence of the target nucleic acid from the second sample of biological material to a complementary single-stranded index sequence bound to a second solid support, wherein the second solid support comprises the same single-stranded index regions as the first solid support;
4. detecting or determining the presence or amount of target nucleic acid from the first sample of biological material relative to the presence or amount of target nucleic acid from the second sample of biological material.

In yet another embodiment, more than one target nucleic acid is amplified from the first sample of biological material and second sample of biological material, wherein each target nucleic acid contains a different index sequence. The solid support may comprise one or multiple single-stranded index regions, for example, 2-200, 50-100, 200-1000 or more index regions, or any integer value subsumed within these ranges.

In any embodiment in which two primers, e.g., P1, and P1 or Pa and Pb, are used to amplify a target nucleic acid, these primers may be used simultaneously or sequentially.

### Definitions

“Target nucleic acids” or “target nucleic acid sequences” both refer to nucleic acid sequences to be detected, with or without amplification. These include the original nucleic acid sequence to be amplified, the complementary second strand of the original nucleic acid sequence to be amplified, and either strand of a copy of the original sequence which is produced by the amplification reaction. These copies serve as amplifiable targets by virtue of the fact that they contain copies of the sequence to which the amplification primers hybridize.

A “labile” or “modifiable” nucleotide is any nucleotide that can be differentially altered with respect to other nucleotides in a polynucleotide such that the polynucleotide becomes susceptible to single-strand cleavage at that site. In some embodiments, the modification process and cleavage occur within the same step. In other embodiments, modification and cleavage may comprise separate steps.

The term “amplicon” refers to the product of the amplification reaction generated through the extension of either or both of a pair of oligonucleotide primers. Amplicons may comprise exponentially amplified nucleic acids if both primers utilized hybridize to a target sequence.

The term “array” refers to a two-dimensional spatial grouping or an arrangement.

The term “microarray” refers to an array of distinct polynucleotides or oligonucleotides synthesized on a substrate, such as paper, nylon or other type of membrane, filter, chip, glass slide, or any other suitable solid support.

A “primer” is a nucleotide sequence used for amplification of a target sequence by extension of the primer after hybridization to the target. The primer is comprised of at least two deoxyribonucleotides or ribonucleotides, preferably at least 5 nucleotides, at least 10 to 15 nucleotides, at least 15 to 30 nucleotides, at least 30-50 or more nucleotides, or any integer value within these ranges. The exact size will depend on many factors, which in turn depends on the ultimate function or use of the primer. The primer may be generated in any manner, including, e.g., chemical synthesis.

“Hybridization” refers to any process by which a strand comprising a polynucleotide binds with a complementary strand through base pairing.

The term “complementary” refers to the natural binding of polynucleotides under permissive salt and temperature conditions by base pairing. For example, the sequence “A-G-T” binds to the complementary sequence “T-C-A.” This is of particular relevance in amplification reactions, which depend upon binding between nucleic acids strands.

The terms “stringent conditions” or “stringency”, as used herein, refer to the conditions for hybridization as defined by the nucleotide sequence, salt concentration, and temperature. These conditions are well known in the art and may be altered in order to identify or detect identical or related polynucleotide sequences. Numerous equivalent conditions comprising either low or high stringency depend on factors such as the length and nature of the sequence (DNA, RNA, base composition), nature of the target (DNA, RNA, base composition), milieu (in solution or immobilized on a solid support), concentration of salts and other components (e.g., formamide, dextran sulfate and/or polyethylene glycol), and temperature of the reactions (within a range from about 5° C. below the melting temperature of the probe to about 20° C. to 25° C. below the melting temperature). One or more factors be may be varied to generate conditions of either low or high stringency different from, but equivalent to, the above listed conditions.

“Amplification,” as used herein, relates to the production of at least one copy of a nucleic acid sequence or its complement. Amplification is generally carried out using, e.g., polymerase chain reaction (PCR) or reverse transcriptase-polymerase chain reaction (RT-PCR) technologies, which are well known in the art. (See, e.g., Dieffenbach, C. W. and G. S. Dveksler (1995) PCR Primer, a Laboratory Manual, Cold Spring Harbor Press, Plainview, N.Y., pp. 1-5.) Nevertheless, as used herein, amplification further comprises any primed nucleic acid synthesis reaction, including, but not limited those mediated by viral, prokaryotic, eukaryotic DNA polymerases, RNA polymerases, and reverse transcriptases.

“Index” or “universal index” sequence refers to a sequence that serves as a reference or standard sequence. This index sequence is incorporated into the 5′ portion of a bipartite primer, which is designed to amplify a specific target nucleic acid, but does not specifically hybridize to the target nucleic acid in the context of the bipartite primer. The index sequence, or index region, of a bipartite primer also contains at least one labile nucleotide for generating a single-stranded region at the terminal end of the double-stranded target nucleic acid. An index sequence may also comprise a nucleic acid probe to bind the single-strand region generated at the terminal end of the double-stranded target nucleic acid. The index region probe may be identical, shorter, longer, or otherwise degenerate from the index region of a corresponding bipartite primer, but must contain sufficiently complementary sequence to specifically bind to the single-stranded region at the terminal end of the double-stranded target nucleic acid. In some embodiments the index region probe is itself bound to a solid support, which may comprise a universal index chip.

As referred to herein, an “amplicon” is a polynucleotide, however, a polynucleotide is not necessarily an amplicon within the context of the invention.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2d ed. 1994); The Cambridge Dictionary of Science and Technology (Walker ed., 1988); and Hale & Marham, The Harper Collins Dictionary of Biology (1991). In addition, the practice of the present invention will employ, unless otherwise indicated, conventional techniques of molecular biology and recombinant DNA technology which are within the skill of the art. Such techniques are explained fully in the literature. See, e.g., Sambrook, Fritsch & Maniatis, Molecular Cloning: A Laboratory Manual, Second Edition (1989).

It is noted that, as used in the specification and the appended claims, the singular forms “a,” “an,” and “the” include plural referents unless the context clearly dictates otherwise. Thus, for example, reference to “a DNA” or “a target nucleic acid” may include two or more such moieties, and the like.

### DETAILED EMBODIMENTS

A. Process for Preparing a Double-Stranded Amplicon Containing a Single-Stranded Region

A process is provided for preparing at least one partially double-stranded target nucleic acid containing at least one single-stranded region at the terminal end. The first primer, P1, comprises at least one labile nucleotide and optionally contains a label. The frequency of labile nucleotides in P1, may be, e.g., about every 3 to 10 bases or every 4 to 5 bases. This ensures that the fragments generated after cleavage will not readily rehybridize to the single-stranded region that is generated by the cleavage. In addition, P1, may contain a labile nucleotide at its 3′ most end, so that all of the primer may be removed upon cleavage and dissociation. The incorporation of the labile nucleotide into the primer may be achieved by including a suitable labile nucleotide in the synthesis reaction mixture for conventional oligonucleotide synthesis. There are many methods for generating oligonucleotide primers, including automated methods.

The second primer, P1, may contain a detectable label or the detectable label may be incorporated into the double-stranded amplicon during amplification. The second primer optionally may contain labile nucleotide(s).

(i) PCR Reaction

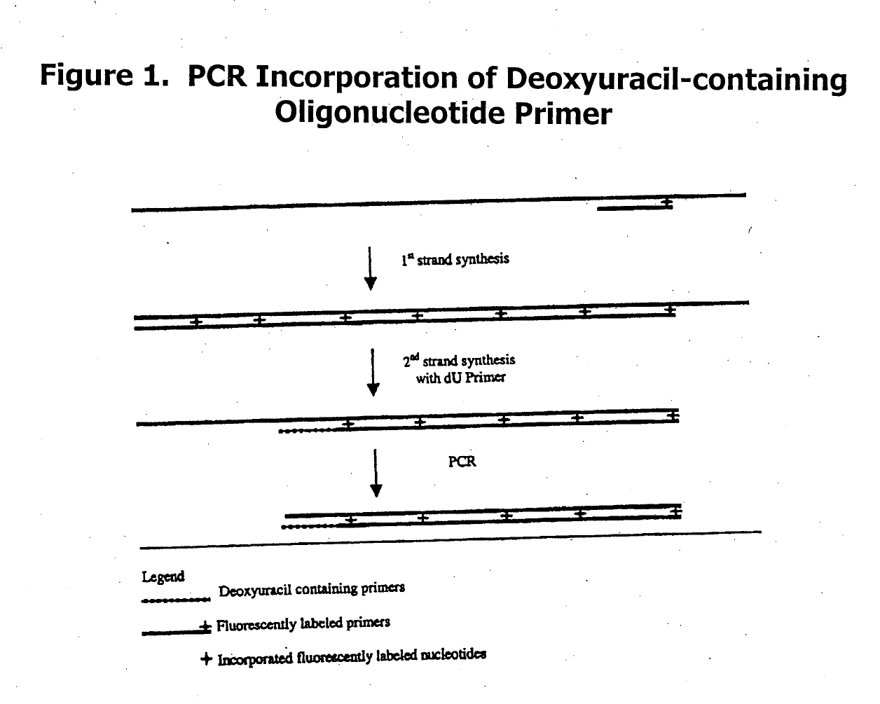

The first and second primers, P1, and P1, may be used in a PCR reaction to generate the double-stranded amplicon from the target nucleic acid containing at least one labile nucleotide. P1, and P1 may be used simultaneously or sequentially as demonstrated in FIG. 1. This amplification reaction is carried out under typical PCR conditions. For example, Applied Biosystems (Foster City, Calif.) provides a kit for RT-PCR utilizing the reverse transcriptase of Thermus thermophilus (rTth DNA polymerase) to amplify the target nucleic acid. In addition to the polymerase, the reaction mixture includes dTTP, dATP, dCTP, and dGTP, buffer, manganese, the chosen primers, and a sample containing the target nucleic acid to be amplified. A sample containing a target mRNA is contacted with P1, under annealing conditions in the presence of a reverse transcriptase and other reagents necessary for primer extension. These other reagents may include, but are not limited to, dNTPs, buffering agents, cationic sources such as KCI and MgCl₂, Rnase inhibitor(s) and sulfhydryl reagents such as dithiothreitol. A variety of enzymes, usually DNA polymerases, possessing reverse transcriptase activity can be used for the primer extension reaction. Examples of suitable DNA polymerases include DNA polymerases derived from organisms such as thermophilic bacteria and archaebacteria, retroviruses, yeasts, insects, primates, and rodents. Preferably, the DNA polymerase is chosen from Moloney murine leukemia virus (M-MLV), M-MLV reverse transcriptase lacking Rnase H activity, human T-cell leukemia virus type 1 (HTLV-1), bovine leukemia virus (BLV), Rous sarcoma virus (RSV), human immunodeficiency virus (HIV) and Thermus aquaticus (Taq) or Thermus thermophilus (Tth), avian reverse transcriptase, and the like. Suitable DNA polymerases possessing reverse transcriptase activity may be isolated from an organism, obtained commercially or obtained from cells which express high levels of cloned genes encoding the polymerases by methods known to those of skill in the art, where the particular manner of obtaining the polymerase will be chosen based primarily on factors such as convenience, cost, availability, and the like.

The order in which the reagents are combined may be modified as desired. One protocol that may be used involves the combination of all reagents except for the reverse transcriptase on ice, then adding the reverse transcriptase and mixing at around 4° C. Amplification may involve, e.g., a 2 min. initial annealing step at 50° C., followed by a 30 minute extension step at 60° C., and a 5 minute deactivation step at 95° C., followed by 40 cycles of 20 sec. denaturation at 94° C. and 1 minute annealing/extending at 62° C. The amplification reaction is then cooled to 4° C. The temperatures and times may be adjusted for optimization for each primer set.

(ii) Labile-Nucleotides and Cleavage Reaction.

Following amplification the double-stranded amplicon is exposed to a reagent that promotes single-stranded cleavage of the amplicon at the site(s) of the labile nucleotide. The cleaved target nucleic acid is then exposed to conditions that promote the dissociation of the cleaved portions of the primer to generate at least one partially double-stranded target nucleic acid containing at least one single-stranded region at the terminal end.

Variations can be made as to the choice of labile nucleotide to be incorporated into the primer, which is used to generate the single-stranded portion of the PCR-generated double-stranded amplicon, as well as the means to subsequently remove the cleaved portions of the primer. The choice of labile nucleotide may include, but is not limited to, the following: (1) RNA bases removed with RNase H, (2) photolabile bases such that cleavage occurs by photolysis, (3) chemically labile linked bases such that cleavage occurs by chemical lysis, (4) primers that contain nucleotides subject to exonuclease activity, but also contain at least one base resistant to such cleavage, e.g., phosphothioate bases, (5) pH sensitive bases, (6) thermolabile bases, etc. The labile nucleotide may also be a substrate for a base-removing enzyme, such that when the labile nucleotide is treated with the enzyme, the incorporated labile nucleotide is removed, generating an abasic site. These abasic sites are subject to cleavage by high temperature or basic pH.

It is well known to the skilled artisan that there are many suitable labile nucleotides for the practice of the present invention. One embodiment utilizes dUTP as the labile nucleotide, which may be cleaved by the base-removing enzyme uridine-N-glycosylase (UNG). Treatment of DNA containing uracil bases (dU) with a uracil DNA glycosylase results in cleavage of the glycosidic bond between the deoxyribose of the DNA sugar-phosphate backbone and the uracil base. The loss of uracil creates an apyrimidinic site in the DNA (Schaaper, R. et al., Proc. Nat. Acad. Sci. USA 80:487 (1983)). The DNA sugar-phosphate backbone that remains after UNG cleavage of the glycosidic bond can then be cleaved by endonuclease IV, alkaline hydrolysis, high temperature, and the like.

When UNG is used to cleave a deoxyuracil-containing oligonucleotide primer, the amount of enzyme used should be sufficient to completely cleave at all dU sites in a reasonable amount of time. For example, 0.5 to 5 units of UNG/100 μl of PCR reaction mixture, preferably 1 to 2 units, is incubated for about 30 minutes at from about 30° C. to about 45° C., preferably at 37° C. The UNG enzyme is inactivated above about 65° C., such that incubating the reaction mixture for about 10 minutes above this temperature, for example at about 95° C. for 10 minutes, will substantially inactivate the enzyme.

When alkaline conditions are to be used to cleave the resulting abasic sites generated by the UNG, approximately 0.1 volume of 0.1 N NaOH (pH of approximately 10-12) is added per 100 μl of reaction mixture and incubated at about 37° C. for 10 minutes. This reaction is neutralized with an equivalent amount of 0.1N HCI acid.

(iii) Labels for Detection

According to a preferred embodiment of the invention, the target nucleic acid may be labeled by at least one method for labeling with a marker for easy detection. As used herein, the terms “label” or “labeled” refer to incorporation of a detectable marker, e.g., by incorporation of a radioactively or non-radioactively labeled nucleotide. These methods of labeling nucleic acid molecules are well known in the art. Labeling may be achieved by incorporating a marker-labeled nucleotide into the PCR product or by incorporating a labeled nucleotide into at least one primer. The following are examples of labels, but are not meant to be an exhaustive representation of the detectable labels that may be used with the present invention.

Examples of radiolabels include, but are not limited to, ³²P, ³H, ¹⁴C, or ³⁵S.

A large number of convenient and sensitive non-isotopic markers are also available. In general, all of the non-isotopic methods of detecting hybridization probes that are currently available depend on some type of derivatization of the nucleotides to allow for detection, whether through antibody binding, or enzymatic processing, or through the fluorescence or chemiluminescence of an attached label molecule. The double-stranded amplicon labeled with non-radioactive labels incorporates single or multiple molecules of the “labeled” nucleotide, generally at specific cyclic or exocyclic positions.

Techniques for attaching non-radioactive groups have largely relied upon (a) functionalization of 5′ or 3′ termini of the monomeric nucleosides by numerous chemical reactions (see Cardullo et al., Proc. Nat'l. Acad. Sci. 85: 8790-8794 (1988)); (b) synthesizing modified nucleosides containing (i) protected reactive groups, such as NH₂, SH, CHO, or COOH, (ii) activatable monofunctional linkers, such as NHS esters, aldehydes, or hydrazides, (iii) affinity binding groups, such as biotin, attached to either the heterocyclic base or the furanose moiety, or (iv) the incorporation of electrochemiluminescent labels such as described by Gudibande et al., Mol. Cell Probes 6(6):495-503 (1992).

According to one embodiment of the invention, the labeled nucleotide(s) are labeled with fluorophores. Examples of fluorophores include fluorescein and derivatives (i.e., isothiocyanate), dansyl chloride, phycoerythrin, allo-phycocyanin, phycocyanin, rhodamine, TEXAS RED® SYBR®-Green (Molecular Probes, Eugene, Oreg.) or other proprietary fluorophores. The fluorophores are generally attached by chemical modification. A fluorescence detector may be used to detect the fluorophores. The label may also include infrared and near infrared dyes.

In another embodiment, the labeled nucleotide can alternatively be labeled with a ligand to provide an enzyme or affinity label. For example, a nucleotide may have biotinyl moieties that can be detected by labeled avidin or streptavidin (e.g., streptavidin containing a fluorescent marker or enzymatic activity that can be detected by optical or calorimetric methods). The enzyme can be peroxidase, alkaline phosphatase or another enzyme giving a chromogenic or fluorogenic reaction upon addition of an appropriate substrate. For example, additives such as 5-amino-2,3-dihydro-1,4-phthalazinedione (also known as LUMINOL® (Sigma Chemical Company, St. Louis, Mo.) and rate enhancers such as p-hydroxybiphenyl (also known as p-phenylphenol) (Sigma Chemical Company, St. Louis, Mo.) can be used to amplify enzymes such as horseradish peroxidase through a luminescent reaction; and luminogeneic or fluorogenic dioxetane derivatives of enzyme substrates can also be used.

The label may also be chosen from a double-stranded DNA selective reagent, e.g., a triple helix or double-stranded DNA intercalating dye.

Usually, the labeled nucleic acid comprises a direct label, such as a fluorescent label, radioactive label, or enzyme-conjugated label that catalyzes the conversion of a chromogenic substrate to a chromophore. One method for detecting the labeled nucleic acid may be Fluorescence Resonance Energy Transfer (FRET), wherein label 1 on either P1, or P1, is excited at wavelength 1 and emits a detectable signal at wavelength 2. A second label may be attached to the other primer or a polynucleotide that binds to any portion of the amplicon, and is excited at wavelength 2, emitting a detectable signal at wavelength 3.

It is also possible, and often desirable for signal amplification, for the labeled binding component to be detected by at least one additional binding component that incorporates a label. Signal amplification can be accomplished by layering of reactants where the reactants are polyvalent.

(iv) Solid Supports for Binding the Target Nucleic Acid

The solid supports for use in the present invention include, but are not limited to, e.g., glass, silicon, plastics such as polycarbonate and polystyrene, porous silicon porous metal oxide, cellulose, nitrocellulose, PVDF, nylon, TEFLON®, polystyrene divinyl benzene, aluminum, carbon, steel, iron, copper, nickel, silver, gold, and other substances suitable for attaching a nucleic acid probe. These materials may be fabricated into tubes, slides, capillaries, microtiter plates, sheets, pins, fibers, beads, chips, or other forms suitable for attaching a nucleic acid probe.

The set of nucleic acid probes bound to the solid support may comprise a DNA microarray. The set of nucleic acid probes may be in the form of discrete spots, each spot representing a specific gene. These spots may also represent a set of index regions (See section C, below).

B. Method for Determining the Presence of a Target Nucleic Acid

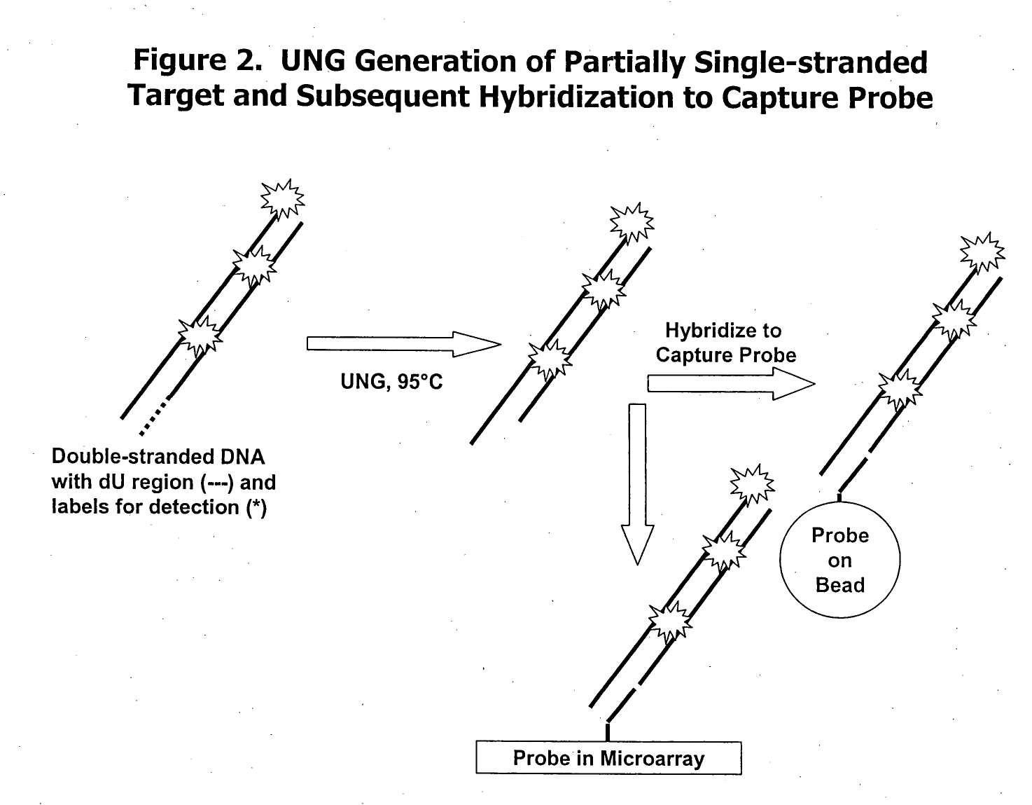

The partially double-stranded target nucleic acid containing a single-stranded region may be used as a way of detecting the presence of a modified target nucleic acid by hybridizing the modified double-stranded amplicon containing a single-stranded region to a set of nucleic acids probes as illustrated in FIG. 2. This set of nucleic acid probes may comprise a nucleic acid microarray, e.g., the Flow-Thru Chip™ (MetriGenix, Inc., Gaithersburg, Md.). Not only can this method be used to detect the presence or absence of a particular target nucleic acid, but it can also be used to detect the relative amount of a particular target nucleic acid.

The method comprises binding a partially double-stranded target nucleic acid containing a single-stranded region to a set of nucleic acid probes, e.g., a DNA microarray, and detecting the presence, absence, or amount of the modified target nucleic acid. The target nucleic acid may be labeled in some way in order to detect its presence.

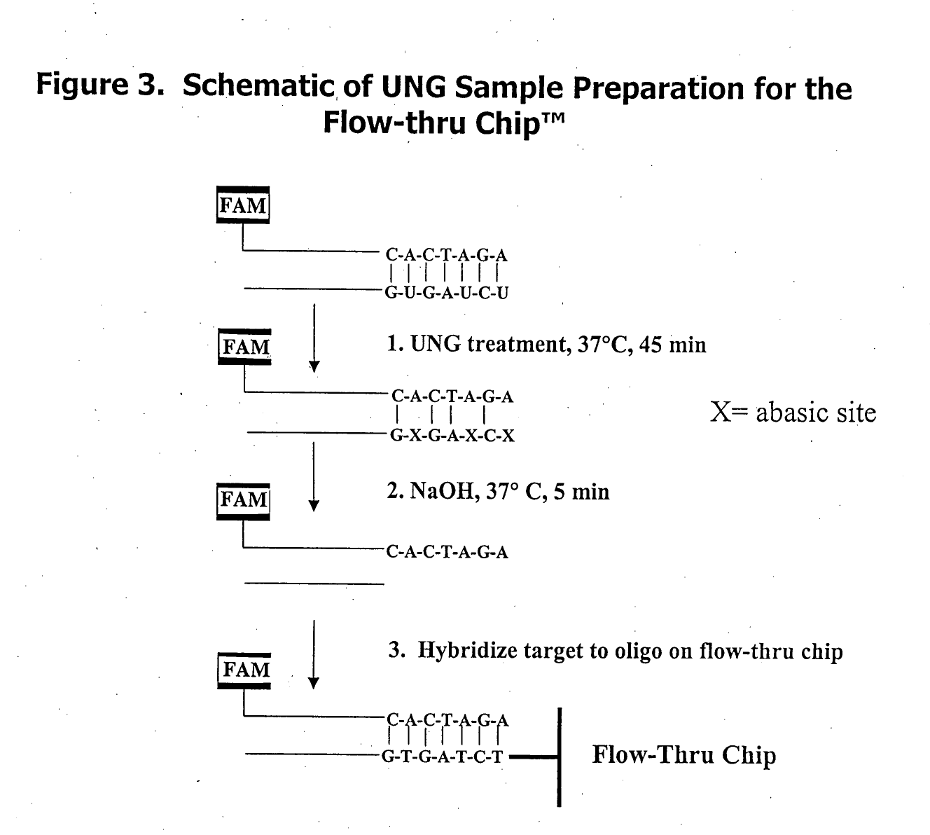

According to a preferred embodiment of the invention, the target nucleic acid may be labeled by at least one method for labeling with a marker for easy detection, e.g., by incorporation of a radioactively or non-radioactively labeled nucleotide. Acceptable labels for detecting these target nucleic acids are described in section A(iii) above. The nucleic acid probes are preferably bound to a solid support. The probes may be in the form of a microarray, e.g., a Flow-Thru Chip™ as illustrated in FIG. 3.

C. Method for Detecting the Relative Amount of a Target Nucleic Acid

One can detect the relative amount of a target nucleic acid by detecting the presence of at least one target nucleic acid isolated from a first sample of biological material relative to the same target nucleic acid isolated from a second sample of biological material. First, at least one modified target nucleic acid is prepared from a first sample of biological material chosen from a given source. This sample of biological material comprises cells that differ from the second sample of biological material to be compared, in, e.g., cell type, tissue type, physiological state, disease state, developmental stage, or drug treatment. For example, the first cell may be chosen from a cancerous cell population and the second cell may be chosen from a normal cell population of the same cell type as the cancerous cell population. Alternatively, the first cell population may have been treated with a pharmaceutical compound, and the second cell population is untreated, serving as a control. The results can provide a gene expression profile useful for in assessing the stage, course, or etiology of a disease, or developing treatment protocols. This expression profile may indicate, e.g., the absence of a particular transcript, the presence of a modified sequence, the overexpression of a particular transcript, or down-regulation of a particular transcript. The presence and/or amount of the partially double-stranded target nucleic acid(s) from the first cell is compared to the same partially double-stranded target nucleic acid(s) from the second cell.

The method comprises contacting at least one partially double-stranded target nucleic acid from the first cell with a first set of nucleic acid probes and contacting at least one partially double-stranded target nucleic acid from the second cell with a second set of nucleic acid probes. The amount of partially double-stranded target nucleic acid from the first cell is compared to the amount of the same partially double-stranded target nucleic acid detected in the second cell. In one embodiment, the partially double-stranded target nucleic acid is synthesized using a bipartite primer having a standardized 5′ end and detected on a universal index chip, as described below.

Acceptable labels for detecting these target nucleic acids are described in section A(iii) above. The nucleic acid probes are preferably bound to a solid support. The probes may be in the form of a microarray, e.g., a Flow-Thru Chip™.

D. Target Nucleic Acid Product

The double-stranded amplicon used in these methods comprises a partially double-stranded target nucleic acid comprising three regions. The first region comprises a single-stranded region of about at least 8 nucleotides or the single-stranded region may be at least 10, 20, or more nucleotides in length. This single-stranded region is generated according to the process described in section A above. The second region comprises that portion of the double-stranded amplicon between the first and third regions. The third region comprises that portion of the double-stranded amplicon comprising P1, and optionally may contain a detectable label.

E. Method for Generating a Target Nucleic Acid Containing an Index Sequence

A method for generating a double-stranded amplicon from a target nucleic acid containing at least one single-stranded index sequence comprises:
1. preparing a bipartite primer, Pa, comprising two regions:
   a) a first region comprising an index sequence and at least one labile nucleotide, wherein the first region is not complementary to the target nucleic acid, and
   b) a second region that is specific for the target nucleic acid;
2. preparing a second primer, Pb, having a sequence specific for the target nucleic acid and optionally containing a detectable label and/or a second index sequence, wherein the optional second index sequence may be the same or different than the index sequence of the first region of Pa;
3. amplifying the target nucleic acid with Pa and Pb generating a double-stranded amplicon;
4. exposing the amplified target nucleic acid to conditions that promote single-stranded cleavage of the amplicon at the site of the labile nucleotide(s); and
5. exposing the cleaved target nucleic acid to conditions that promote the dissociation of the cleaved portions of the primer containing the labile nucleotide to generate a single-stranded region at the terminal end of the target nucleic acid.
   F. Method for Detecting the Presence or Amount of a Target Nucleic Acid Using a Universal “Index” Chip

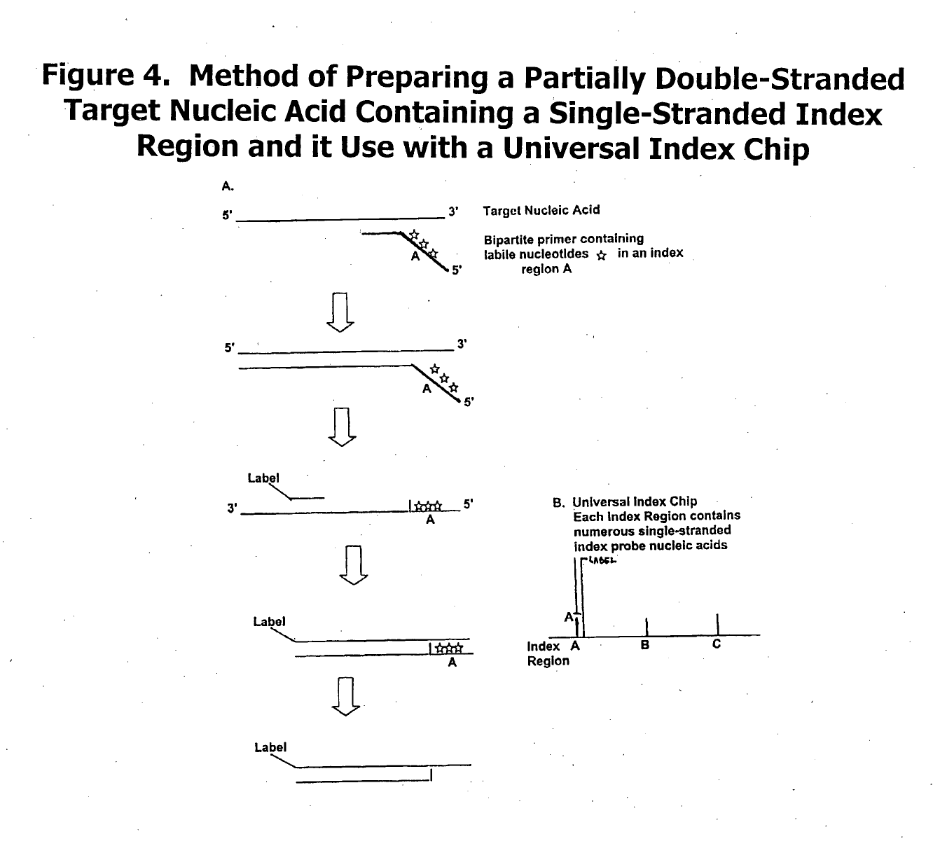

There is provided a method for determining the presence of a target nucleic acid in a first sample of biological material relative to a second sample of biological material by incorporating the same “index” sequence into the same target nucleic acid of the first and second samples as illustrated in FIG. 4. Labeled partially double-stranded target nucleic acids from the first and second samples are separately hybridized to a probe affixed to a solid support and comprising sufficient nucleic acid sequence to hybridize to the index region of the modified target. Detecting or determining the signals from the labeled nucleotides and correlating these values to the amount of target sequence in the first and second samples of biological material measures the relative amounts of bound target nucleic acid.

Similarly, multiple targets may be simultaneously compared by employing multiple bipartite primers each having different index and target portions. The labeled targets synthesized using these primers are detected or determined by hybridization to multiple index regions, each corresponding to an index region of one of the bipartite primers.

Thus, the presence or amount of a target nucleic acid sequence (or sequences) in a first sample of biological material relative to the same nucleic acid sequence (or sequences) in a second sample of biological material may be assayed by:
1. preparing at least one bipartite primer, Pa, comprising two regions:
   a) a first region comprising a first index sequence and at least one labile nucleotide, wherein the first region is not complementary to the target nucleic acid sequence, and
   b) a second region that is complementary to the target nucleic acid sequence;
2. preparing a second primer, Pb, having a sequence specific for the target nucleic acid sequence and optionally containing a detectable label and/or a second index sequence, wherein the optional second index sequence may be the same or different than the index sequence of the first region of the Pa;
3. amplifying a specific target nucleic acid sequence from a first sample of biological material to generate a double-stranded amplicon;
4. amplifying a specific target nucleic acid sequence-from a second sample of biological material to generate a double-stranded amplicon;
5. exposing the amplicons to conditions that promote single-stranded cleavage of the amplicons at the site(s) of the labile nucleotides;
6. exposing the cleaved amplicons to conditions that promote the dissociation of the cleaved portions of the index region of the primer to generate a single-stranded region at the terminal end of the target nucleic acids;
7. hybridizing the single-stranded index sequence of the double-stranded amplicons from the first sample of biological material to a complementary single-stranded index sequence bound to a first solid support, wherein the solid support may comprise at least one single-stranded index region;
8. hybridizing the single-stranded index sequence of the double-stranded amplicons from the second sample of biological material to a complementary single-stranded index sequence bound to the first solid support or a second solid support, wherein the second solid support may comprise the same single-stranded index regions as the first solid support; and
9. detecting or determining the amount of double-stranded amplicon from the first sample of biological material relative to the double-stranded amplicon from the second sample of biological material.

In the above embodiment, if the second primer, Pb, also contains an index sequence, it may be the same or different than the index sequence of the first region, and if present, must contain at least one labile nucleotide in order to generate a single-stranded region. Alternatively, the second primer may contain a different nucleic acid sequence that does not bind to the target nucleic acid sequence, as long as that sequence is not able to bind to the same region of the solid support as occupied by the index region probe. This alternative second sequence can be used to bind, e.g., a labeled nucleic acid fragment.

These two primers, Pa and Pb, are used to amplify the target nucleic acid generating a double-stranded amplicon, and as described above, a single-stranded region is generated upon cleavage of the labile nucleotide and dissociation of the cleaved portions from the target nucleic acid. In this instance, the single-stranded region comprises sufficient nucleic acid sequence to bind to an index region on the solid support.

The single-stranded index sequence of the double-stranded amplicons from the first sample of biological material may be hybridized to a complementary single-stranded index sequence bound to a first solid support. The solid support comprises at least one single-stranded index region. The single-stranded index sequence of the double-stranded amplicons from the second sample of biological material bind to a complementary single-stranded index sequence bound to a second solid support, wherein the second solid support comprises the same single-stranded index regions as the first solid support. Alternatively, the single-stranded index sequence of the double-stranded amplicons from the second sample of biological material may be hybridized to complementary single-stranded index sequence bound to the first solid support. In this instance, the label incorporated into the target nucleic acid from the second sample must be different from the label incorporated into the target nucleic acid from the first sample.

The next step involves detecting or determining the presence/amount of double-stranded amplicon from the first sample relative to the double-stranded amplicon from the second sample. More than one target nucleic acid may be amplified from the first and second samples, wherein each target nucleic acid amplified from the first sample contains a different index sequence. The same set of target nucleic acids would be amplified from the second sample and would contain the same respective index sequences.

The solid support used in the detection may comprise multiple single-stranded index regions, for example, 2-1000 index regions, 50-100, 100-500, 500-10,000, or more, index regions, or any range of integers subsumed within these ranges. The solid support may be a DNA chip having multiple index regions.

Use of an index sequence allows the construction of a universal “index” chip having one or more index region probes. Irrespective of the target nucleic acid sequence one wants to detect, the same chip may be used, merely by programming the index corresponding to the probe into a bipartite primer. For example, Researcher in Laboratory A wishes to study the expression of C-myc in a tumor cell-line. Researcher in Laboratory B wishes to study the developmental regulation of a Xenopus cell. Both laboratories can use the same universal “index” chip by incorporating index sequence “A” into their specific target nucleic acid and hybridizing the target nucleic acid to index region A on the universal “index” chip. Each researcher can look at, e.g., 1000 different expressed sequences, by incorporating a different index sequence into each different target nucleic acid. It does not matter that the sequences that researcher A examines are completely different from the sequences researcher B is examining. They may still use the same universal “index” chip, because the bipartite primer contains the necessary index sequence in combination with the sequence specific for the desired target nucleic acid.

G. Drug-Screening Using a Target Nucleic Acid Containing a Single-Stranded Region

Another aspect of the invention utilizes the speed and sensitivity of analyses of the Flow-Thru Chip™, which makes high-throughput multiple-gene screening a viable possibility. Multiple-gene screening can be used as a tool to screen pharmaceutical candidates for both drug efficacy and toxicity, either separately or in tandem. In a multiple-gene drug-screening assay, the change in expression of several gene targets is monitored as a function of drug composition, dose, and time. Gene targets are selected on the basis of known association with the disease target of the pharmaceutical compound or known toxicological side-effects. (See Example 4) Parallel analysis of multiple target nucleic acid (e.g. 50-100, 100-500, 500-10,000, or more) should prove quite valuable in the determination of viable drug targets from a pharmaceutical compound library (Amundson, S. et al., Oncogene 18:3666-3672 (1999); Kahn, J., et al., Electrophoresis 20:223-229 (1999)). Effective pharmaceutical agents are selected on the basis of their ability to move a ‘diseased’ gene expression profile to a ‘normal’ profile; whereas toxic agents are determined when a toxicity gene panel profile is modified from the ‘normal’ profile.

H. Microbial Detection and Classification using Target Nucleic Acids Containing a Single-Stranded Region

Sensitive detection of microbial agents can be attained quickly and easily via PCR of genomic DNA in samples. The samples may be of clinical or environmental relevance. Microbial agents under study may include, for example, strains of Staphylococcus aureus, Influenza, Mycobacterium Tuberculosis. Not only may the presence of one of these agents be determined, but the antiobiotic resistance of the present agent can be classified as well. For example, in Mycobacterium Tuberculosis there is a strong correlation between in-vitro resistance to rifampicin and pyrazinamide and mutations in rpoB and pncA, respectively (Brown, T. J. et al., J. Med. Microbiol. 49:651-656 (2000)).

Rapid detection and classification could be achieved by designing primers for each strain to be tested as well as for genes which are associated with antibiotic resistance. Primers can be designed with the reverse primer including a labile nucleotide and the forward primer containing a 5′ biotin. Microrganisms may be detected and amplified by amplification of genomic DNA sequences from genomic DNA isolates using the PCR method as described in Detailed Embodiment A followed by hybridization of the resulting single-stranded overhang-containing amplicons to a microarray. In a preferred embodiment, the microarray is a Flow-thru Chip™. Hybridized single-stranded overhang-containing amplicon may be detected by staining the chip with a streptavidin-horseradish peroxidase conjugate (Pierce Endogen, Rockford, Ill.) and developing with TMB liquid substrate (Sigma Chemical, St. Louis, Mo.) which results in a purple spot.

### DETAILED EXAMPLES

The following Examples are merely illustrative and not intended to be limiting in any way.

### Example 1

### Sample Preparation

A sample containing the target nucleic acid was prepared as follows. RNA was isolated using the RNeasy kit (Qiagen, Valencia, Calif.) using the manufacturer's recommended conditions. This method is suitable for isolating up to 100 μg of RNA, which is the approximate binding limit of the RNeasy mini spin column. All buffers mentioned below are provided with the RNeasy kit. The Buffer RLT was warmed to dissolve any precipitate, and then β-mercapto-ethanol (“BME”) (10 μl per 1 ml of Buffer RLT) was added before use. Four volumes of 100% EtOH also was added to Buffer RPE before initial use. The sample (lysed and digested cells or tissue that is deproteinated and delipidated) was adjusted to 100 μg/100 μl using RNase-free H₂O. If the sample was more than 130 μl, it was split into two tubes, and each was diluted to 100 μl with RNAsecure. Samples were placed into a 1.5 ml tube(s), and 350 μl of Buffer RLT was added, with mixing. Then 250 μl of 100% EtOH was added with mixing by pipetting. The sample (approx. 700 μl) was added to the RNeasy Column, which was centrifuged (spun) at room temperature for 15 seconds at 10,000 rpm.

The sample from the collection tube was reapplied to the same column, respun for 15 seconds at 10,000 rpm, and transferred to a new collection tube. 500 μl of Buffer RPE was added and the sample spun at room temperature for 15 seconds at 10,000 rpm to wash. An additional 500 μl of Buffer RPE was added to the column and spun at maximum speed to dry the membrane within the column.

The column was transferred to a new 1.5 ml collection tube, and 30 μl of DEPC H₂O was added directly onto the membrane. After a 5 minute incubation, the sample was spun for 1 minute at 10,000 rpm to elute. The eluate (30 μl) was added back to the column and spun again at 10,000 rpm. The OD of the final eluate was determined and the ratio of absorbance at 260 and 280 nm (“280/260 ratio”) was determined and used to calculate the concentration of RNA using standard methods. A total RNA yield of between 0.5 μg and 5.7 μg was obtained before proceeding to cDNA synthesis either before or after total RNA cleanup. This sample was diluted to 1 mg/ml using DEPC water. If the concentration of the RNA was too low, it first was precipitated using standard methods followed by redilution to 1 mg/ml.

For mRNA preparations, 1-5 μg of poly(A)+ RNA is used (5 μg being preferred for some applications) for the first primer extension reaction; for the second strand synthesis, only about 1.5 μg of primer, is added. For total RNA (which contains structural RNA plus mRNA) more starting RNA is preferred, e.g. 5-40 μg, typically 25-30 μg, and 1.5 μg of second strand primer is used.

### Example 2

### UNG Degradation of Forward Primer

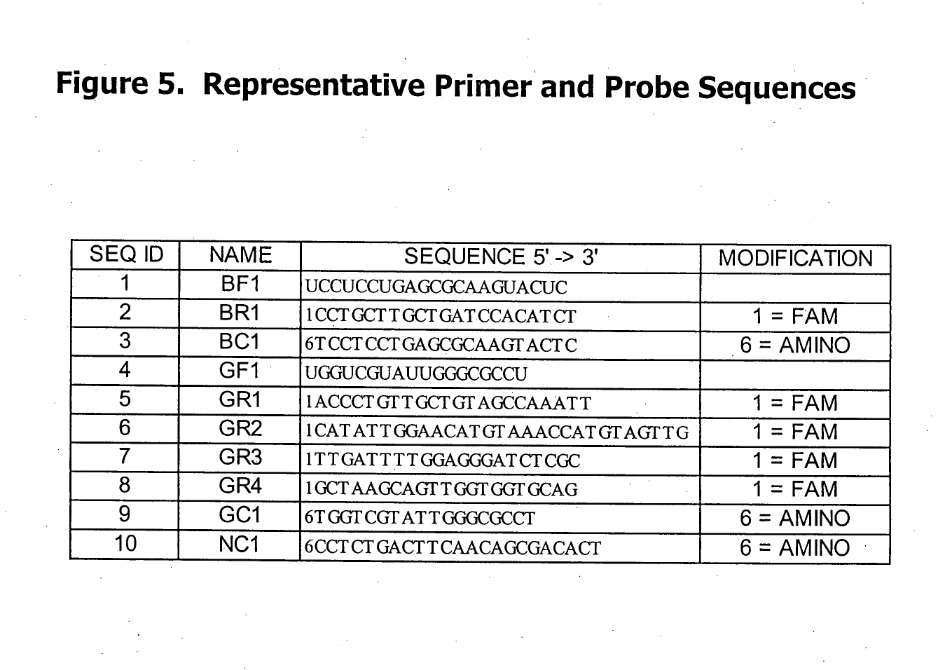

The degradation of the dU-containing primer was assessed by performing a gel shift assay. Primers and probes were synthesized by standard synthesis procedures. A listing of primers and probes is given in FIG. 5. Double stranded amplicons were produced from a forward dU-containing primer and a reverse non-dU containing primer. The primers were incorporated into a double-stranded DNA molecule using a thermocycler according to the protocol in Table 1 and using RNA obtained by methods described in Example 1. The components used in the PCR amplification reaction can be found in Table 1. RT-PCR amplification was done according to the instructions of the thermocycler used, e.g. PERKIN ELMER GeneAmp PCR system. Optimization guidelines are provided with commercially available RT-PCR kits to adjust conditions to obtain the highest yield of RT-PCR product. Following amplification, samples were cooled to 4° C.

TABLE 1
RT-PCR Reaction Components

| | Volume | Concentration | |
|---|---|---|---|
| Component | (μL) | (Final) | |
| Rnase-free water | 14 | — | |
| 5× Buffer | 10 | 1× | |
| Manganese Acetate | 6 | 3 | mM |
| dATP (10 mM) | 1.5 | 300 | μM |
| dGTP (10 mM) | 1.5 | 300 | μM |
| dCTP (10 mM) | 1.5 | 300 | μM |
| dTTP (10 mM) | 1.5 | 300 | μM |
| Forward Primer (dU) | 1 | 1000 | nM |
| Reverse Primer | 1 | 1000 | nM |
| RNA (3 ng/μL) | 10 | 30 | ng |
| Polymerase | 2 | 0.1 | U/μL |

In the gel shift assay, a probe capable of hybridizing to the single stranded overhang regions was presented to amplicon that had been treated under a variety of treatments including UNG and base. A radioisotope labeled (33P) aliquot of BC1 was added to each of 5 tubes with components according to Table 2, lines 1-3. The initial mixture, lines 1-3, was incubated at 37° C. for 30 minutes. The indicated volume of NaOH was added per line 4 of Table 2 and the tube was incubated at 37° C. for an additional 10 minutes. The indicated volume of HCl was added per line 5 of Table 2.

TABLE 2
Gel Shift Assay Tube Components

| | | Tube | | | | | | | | | |
|---|---|---|---|---|---|---|---|---|---|---|---|
| Line | Component | 1 | | 2 | | 3 | | 4 | | 5 | |
| 1 | BC1 | 100 | pmol | 100 | pmol | 100 | pmol | 100 | pmol | 100 | pmol |
| 2 | Amplicon | 100 | pmol | 100 | pmol | 100 | pmol | 100 | pmol | 100 | pmol |
| 3 | UNG | 0 | | 0 | | 1 | unit | 1 | unit | 0 | |
| 4 | 0.1 N NaOH | 0 | | 0 | | 0 | | 10 | μL | 10 | μL |
| 5 | 0.1 N HCl | 0 | | 0 | | 0 | | 10 | μL | 10 | μL |

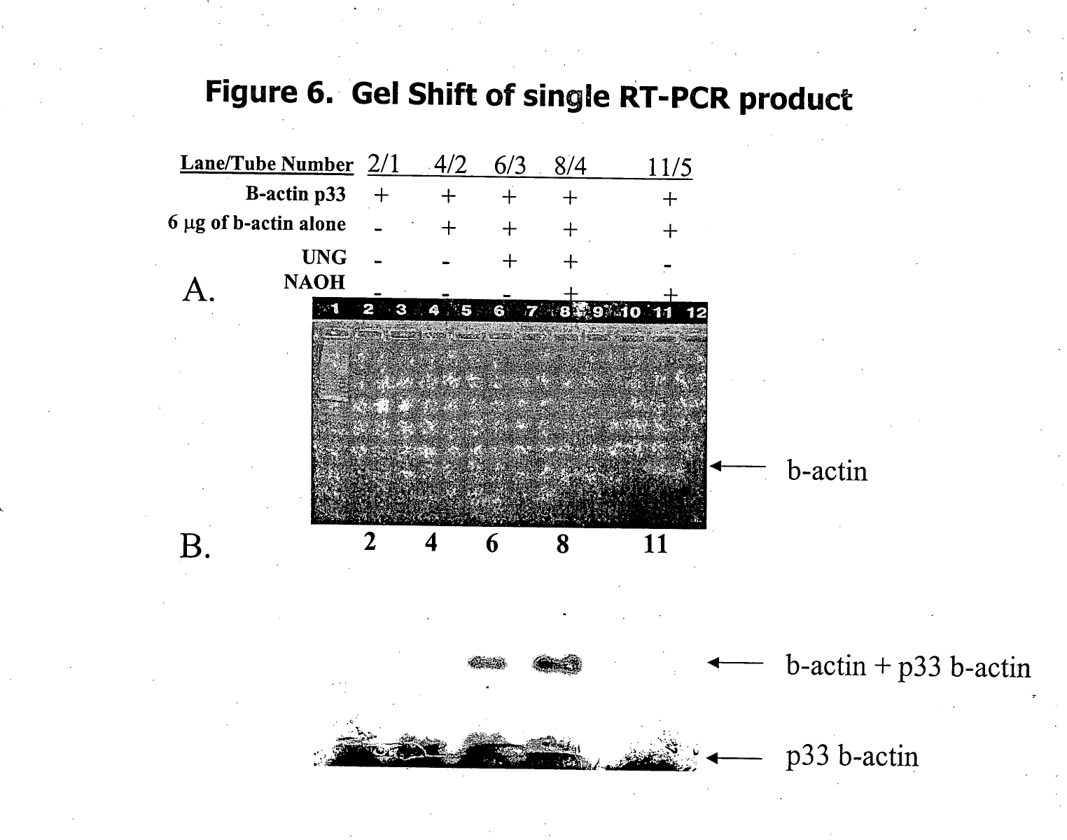

According to the reagent mixtures in the 5 tubes, the assay should only show a shift in migration of the labeled BC1 for tube number 4. An image of the gel shift is given in FIG. 6. Bands are visible for tubes 3 and 4; however, the intensity of tube 4 is roughly 10 times higher than that of tube 3. The gel shift observed suggests that UNG alone is insufficient to degrade the labile primer and that the combination of UNG and cleavage, by base in this example or temperature, is necessary.

### Example 3

### Generation of Multiplex Samples

Two different target nucleic acids were chosen for modification and amplification. The target nucleic acids amplified were human β-actin and GAPDH. The β-actin target was produced from primers (BF1, BR1) that produced a 97 base pair amplicon. For GAPDH, a single forward dU-containing primer (GF1) and 4 reverse primers were designed to give double stranded amplicons of 117(GR1), 123(GR2), 226(GR3), and 442(GR4) base pairs. All of the reverse primers included a fluorescent FAM dye at the 5′ end of the sequence. The primers were incorporated into a double-stranded DNA molecule either in singleplex or in duplex using a thermocycler according to the following protocol.

TABLE 3
Multiplex PCR Preparation Components

| | Tube/Lane | | | | | |
|---|---|---|---|---|---|---|
| | 1 | 2 | 3 | 4 | 5 | 6 |
| | Volume | Volume | Volume | Volume | Volume | Volume |
| Component | (μL) | (μL) | (μL) | (μL) | (μL) | (μL) |
| Rnase-free | 14 | 14 | 14 | 14 | 14 | 14 |
| water | | | | | | |
| 5× Buffer | 10 | 10 | 10 | 10 | 10 | 10 |
| Manganese | 10 | 10 | 10 | 10 | 10 | 10 |
| Acetate | | | | | | |
| dNTP (N = A, | 2 | 2 | 2 | 2 | 2 | 2 |
| G, C, T | | | | | | |
| @ 10 mM) | | | | | | |
| BF1 (50 nM) | 1 | 0 | 0 | 0.5 | 0.5 | 0.5 |
| BR1 (50 nM) | 1 | 0 | 0 | 0.5 | 0.5 | 0.5 |
| GF1 (50 nM) | 0 | 1 | 1 | 0.5 | 0.5 | 0.5 |
| GR1 (50 nM) | 0 | 1 | 0 | 0.5 | 0 | 0 |
| GR2 (50 nM) | 0 | 0 | 1 | 0 | 0 | 0 |
| GR3 (50 nM) | 0 | 0 | 0 | 0 | 0.5 | 0 |
| GR4 (50 nM) | 0 | 0 | 0 | 0 | 0 | 0.5 |
| RNA (3 ng/μL) | 10 | 10 | 10 | 10 | 10 | 10 |
| Polymerase | 2 | 2 | 2 | 2 | 2 | 2 |

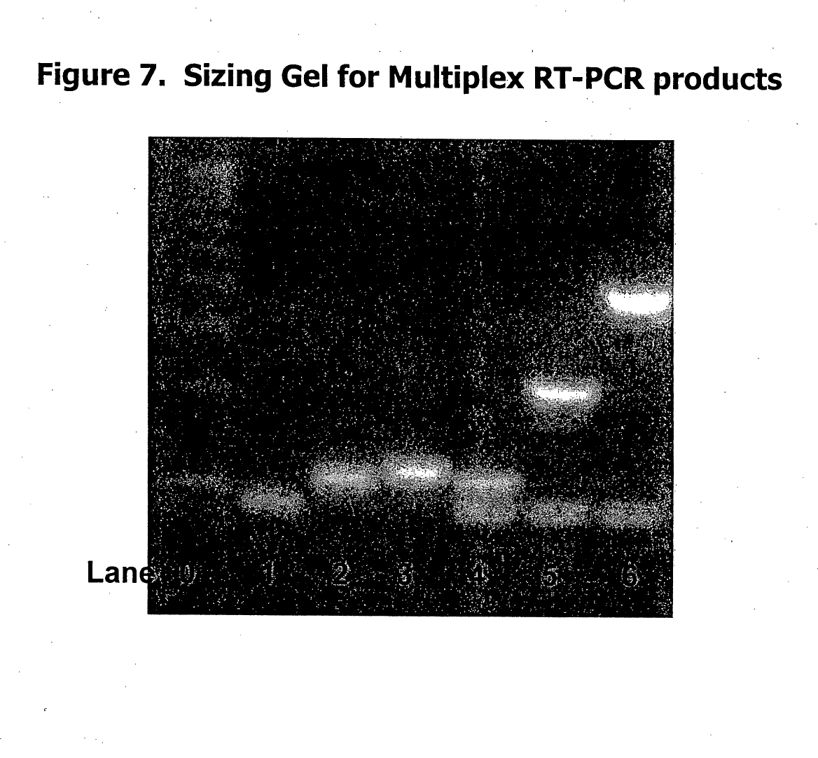

The PCR amplicons were run on a sizing gel to determine the number of bands in each lane and the size of the amplicons. A picture of the gel is given in FIG. 7 with the lane label corresponding to the PCR tube in Table 3. Each reaction that was supposed to yield 2 bands did so, with each band being the appropriate size as well.

The PCR amplicons from Tubes 1,2, and 4-6 were treated according to the protocol for Tube 4 in Example 2 to degrade the dU-containing portion and generate a single stranded overhang. The resulting partially double-stranded amplicons were hybridized to a Flow-thru Chip™ in 1×SSPE buffer for 1 hour. The Flow-thru Chip™ contained a 4×3 array of probes where 3 unique probes (BC1, GC1, and NC1) were spotted in quadruplicate by row as indicated in Table 4. The signal intensity values for each row are given in Table 4 as well. The data indicate minimal cross-hybridization between the individual components (1&2), and nominal dependence of relative hybridization efficiency on amplicon size in the range of 97 to 442 base pairs (4-6).

TABLE 4
Flow-thru Chip Results ™

| | | Tube | | | | |
|---|---|---|---|---|---|---|
| Row | Probe | 1 | 2 | 4 | 5 | 6 |
| 1 | BC1 | 315 | 5 | 354 | 205 | 279 |
| 2 | GC1 | 6 | 389 | 414 | 355 | 216 |
| 3 | NEG | 4 | 5 | 6 | 5 | 6 |
| | RATIO (B/G) | 53 | 0.02 | 1.2 | 1.7 | 0.8 |

### Example 4

### Analysis of Gene Expression Patterns

MCF-7 cells are estrogen responsive breast adenocarcinoma cells that have been used previously as a model system for evaluation of breast cancer drugs. Initial studies have examined the effect of an anti-estrogen, tamoxifen, on the estradiol response of MCF-7 cells. Estradiol stimulates cell proliferation through the G1 phase in synchronized cell growth studies. Tamoxifen treatment of cells inhibits the induction of several genes that occur upon estradiol treatment by blocking estrogen receptor cofactors required for transcription. The Flow-Thru Chip™has been used as an analysis platform for a set of genes reported in the literature as differentially regulated in the MCF-7 system under estradiol and tamoxifen treatment. TAQMAN® PCR was performed to measure the changes in expression levels for each of the monitored genes across the treatments.

The experimental protocols for estradiol and tamoxifen treatment have been described in detail elsewhere (Wosikowski et al., Int. J. Cancer 53:290-297 (1993)). In this study MCF-7 (ATTC, Manassas, Va.) cells were maintained in MEM alpha medium supplemented with 10% heat-inactivated fetal bovine serum, Penicillin-Streptomycin and 10 μg/ml bovine insulin (complete medium) (Life Technologies, Gaithersburg, Md.). For the treatment experiment, 1×10⁵ cells/cm² were plated into T75 flasks, using 2 ml per 10 cm² complete medium and allowed to attach for 24 hours. During the next 3 days the cells were rinsed twice with PBS and incubated with MEM phenol-free medium supplemented with 5% dextran sulfate/charcoal stripped fetal bovine serum, Penicillin-Streptomycin, L-glutamine, 10 μg/ml bovine insulin, and 1×MEM non-essential amino acids (stripped medium). The stripped medium was changed daily for two more days. Four samples were prepared: a vehicle-treated control, an estradiol treatment, a tamoxifen treatment, and a mixed estradiol and tamoxifen treatment. The cells were incubated for 6 hours with stripped medium in the presence of 10 nM 17-beta-estradiol, 1 μM tamoxifen, and a combination of 10 nM 17-beta-estradiol with 1 μM tamoxifen. Total RNA was harvested using the RNeasy kit (Qiagen, Valencia, Calif.) according to manufacturer's instructions. The purity and integrity of the total RNA was assessed by the 260/280 absorbance and the 28s/18s RNA ratios.

Analysis of genes under test by the Flow-Thru Chip™ required amplification from the total RNA. Selective amplification was performed using multiplex RT-PCR. Amplification was performed using 60 ng of total RNA and an EZ RT-PCR kit (PE Applied Biosystems, Foster City, Calif.). Genes were grouped into four multiplex pools according to the relative abundance in the sample and a different number of PCR cycles were performed for each pooling. The appropriate number of cycles for each grouping of target nucleic acids was determined empirically to assure that all targets in the group were in the linear dynamic range of the chip and had not reached primer limitation in the PCR amplification. Each pool contained 5 to 8 target nucleic acids and a few target nucleic acids were amplified in more than one multiplex reaction.

The ability to multiplex target nucleic acids under study was confirmed by TAQMAN®, analysis of the singleplex and multiplex amplifications. Detection of the amplified products on the chip was afforded by a fluorescent tag incorporated at the 5′ end of one of the primers for each gene. Amplicons were captured by probes on the chip via single-stranded region generated according to the methods described above. Flow-Thru Chips™ were prepared with probes for 12 target nucleic acids, including 3 controls. Probe sequences were complementary to the single-stranded region unique to each target. Changes in expression level between samples were determined by comparing the brightness of gene-spots measured for each target nucleic acid relative to a reference target nucleic acid, β-actin. The same reference nucleic acid was used in the TAQMAN® analysis to determine changes in expression.

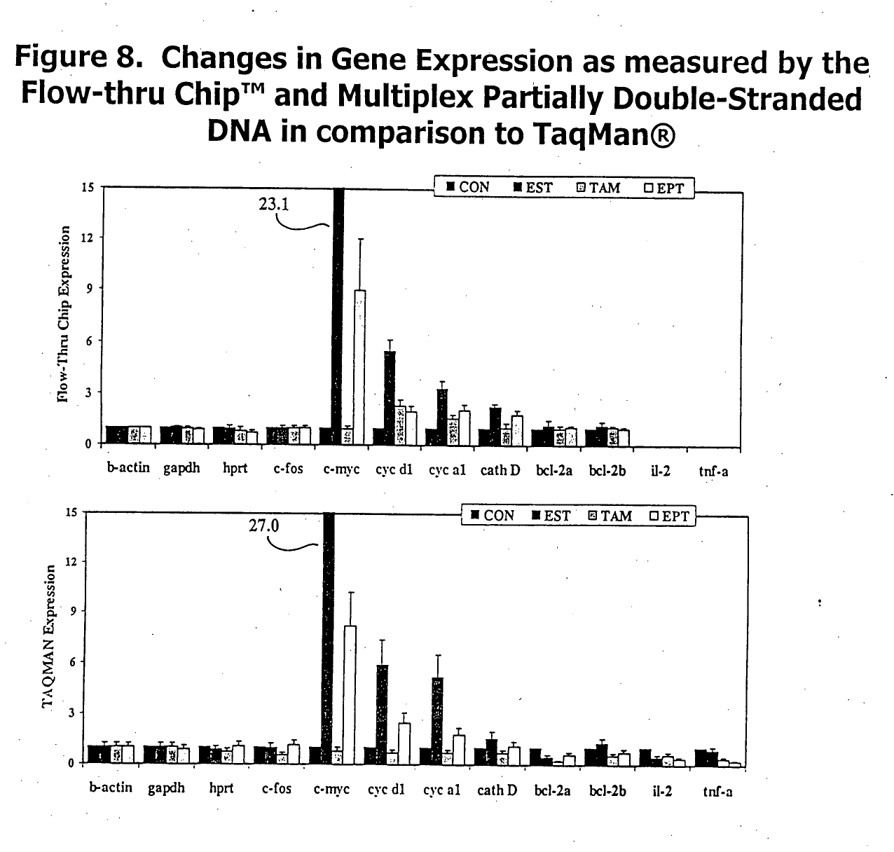

The change in expression level of the 12 target nucleic acids in this study across the four cell treatments, as determined by the Flow-Thru Chip™ and TAQMAN®, are presented in FIG. 8. The target nucleic acids included c-fos, c-myc, cyc D1, cyc A1, cath D, bcl-2a, bcl-2b, IL-2, TNF-α, and three controls, β-actin, GADPH, HPRT. The signal measured for each target nucleic acid in the control sample was set to an expression level of 1.0 and changes in expression for the other three samples were determined relative to reference signal. In general, the Flow-Thru Chip™ and TAQMAN® results were in very good quantitative agreement.

As previously disclosed in the literature, estradiol induces expression of a set of target nucleic acids and tamoxifen, when added to the estradiol, suppresses the induction for this set of target nucleic acids. Tamoxifen alone has little influence on the expression of any of the target nucleic acids included in this study. β-actin, a so-called ‘housekeeping’ gene, was used as the reference gene. Two other housekeeping genes were included glyceraldehyde-3-phosphate dehydrogenase (GAPDH) and hypoxanthine phosphoribosyl transferase (HPRT) and showed no variation in expression across the four cell treatments.

C-fos is an early transcription factor and thereby was not expected, nor was it observed to be differentially regulated at 6 hours after treatment.

C-myc is an early to intermediate estrogen responsive gene. C-myc was measured as 23±4 and 27±6 fold up-regulated in the estradiol treatment by the Flow-Thru Chip™ and TAQMAN®, respectively. Tamoxifen alone had no influence on C-myc, but in conjunction with estradiol, inhibited up-regulation to a roughly 8-fold increase, as determined by both methods.

Cyclin D1 (cyc D1) and cyclin A1 (cyc A1) are associated with cell growth. Cathepsin D (cath D) is a protease associated with metastasis that has been identified as a prognostic factor in breast cancer. While the measurement variability suggests that the roughly 2-fold induction of the Cathepsin D is a real change, such small fold changes in expression are not routinely considered significant in PCR-based methods. BCL-2 alpha (bcl-2a) and beta (bcl-2b) block apoptosis. Interleukin-2 (IL-2) and tumor necrosis factor alpha (TNF-a) were determined in very low abundance in the MCF7 cells by TAQMAN®, but were not determined above the background on the Flow-Thru Chip™.

The data presented in FIG. 8 required 4 individual chip runs to acquire the expression level changes for the 12 target nucleic acids. Conditions can be optimized such that multiple RT-PCR preparations can be combined and run over a single chip. This initial small-scale study suggests that the Flow-Thru Chip™is a viable readout platform for a substantial screening program with a larger gene set, drug set, and temporal dosage regimen.

Each reference cited herein is hereby incorporated by reference in its entirety.

1. A process for generating at least one partially double-stranded polynucleotide containing at least one single-stranded region at a terminal end prepared by:
a) providing at least one primer, P1, containing at least one labile nucleotide;
b) combining at least one target nucleic acid sequence with P1, to generate a double-stranded polynucleotide containing at least one labile nucleotide;
c) exposing the double-stranded polynucleotide to conditions that promote single-strand cleavage of the polynucleotide at the site of the at least one labile nucleotide of primer P1; and
d) exposing the cleaved polynucleotide to conditions that promote the dissociation of the cleaved portions of primer P1, from a terminal end.

2. The process according to claim 1, wherein the labile nucleotide is dUTP, and wherein the single-stranded cleavage of the polynucleotide at the site of the labile nucleotide occurs by treatment with uracil N-glycosylase.

3. The process according to claim 1, wherein the target nucleic acid sequence is chosen from mRNA, cDNA, genomic DNA, recombinant DNA, plasmid DNA, and amplified DNA.

4. The process according to claim 1, wherein part (b) involves the amplification of a single target nucleic acid sequence.

5. The process according to claim 1, wherein part (b) involves the amplification of 2-1000 target nucleic acid sequences.

6. The process according to claim 1, wherein at least one labile nucleotide is chosen from 5-hydroxy-2′deoxycytidine triphosphate, 5-hydroxy-2′deoxyuridine triphosphate, RNA, a photolabile base, a thermolabile base, a pH sensitive base, a chemically labile base, and an exonuclease sensitive base provided that when exonuclease is used the primer also contains at least one phosphothioate base.

7. The process according to claim 1, wherein the partially double-stranded polynucleotide contains at least one detectable label.

8. The process according to claim 7, wherein the detectable label is chosen from a radioisotope, a chromophore, a fluorophore, an enzyme, an antigen, a reactive group, and a double-stranded DNA selective reagent.

9. The process according to claim 1, wherein P1, comprises 5 to 50 nucleotides.

10. The process according to claim 9, wherein P1, comprises 10 to 30 nucleotides.

11. The process according to claim 1, wherein a second primer, P1, is used in the generation of the double-stranded polynucleotide.

12. The process according to claim 11, wherein P1 contains a detectable label.

13. The process according to claim 11, wherein P1 comprises 5 to 50 nucleotides.

14. A partially double-stranded target nucleic acid containing at least one single-stranded region at the terminal end prepared according to claim 1.

15. A method for detecting the presence or determining the amount of a target nucleic acid sequence comprising:
(a) preparing at least one partially double-stranded polynucleotide containing a target nucleic acid sequence and at least one single-stranded region at a terminal end according to the process of claim 1,
(b) hybridizing the partially double-stranded polynucleotide to one or more sets of nucleic acid probes, and
(c) detecting the presence or determining the amount of the hybridized partially double-stranded polynucleotide.

16. The method according to claim 15, wherein the partially double-stranded polynucleotide contains at least one detectable label.

17. The method according to claim 16, wherein the detectable label is chosen from a radioisotope, a chromophore, a fluorophore, an enzyme, an antigen, a reactive group and a double-stranded DNA selective reagent.

18. The method according to claim 15, wherein one or more sets of nucleic acid probes is attached to a solid support.

19. The method according to claim 18, wherein the solid support is chosen from capillary tubes, beads, fibers, slides, sheets, pins, microtiter plates, silicon, porous silicon, porous metal oxide, plastic, polycarbonate, polystyrene, cellulose, nitrocellulose, nylon, PVDF, glass, TEFLON®, polystyrene divinyl benzene, aluminum, carbon, steel, iron, copper, nickel, silver, and gold.

20. The method according to claim 15, wherein one or more sets of nucleic acid probes comprise a DNA microarray.

21. A method of detecting or determining the presence or amount of at least one target nucleic acid sequence in a first sample of biological material relative to the same target nucleic acid sequence(s) in a second sample of biological material comprising:
(a) preparing at least one partially double-stranded polynucleotide containing a target nucleic acid sequence and at least one single-stranded region at a terminal end for each target nucleic acid sequence from both the first sample of biological material and the second sample of biological material, according to the process of claim 1;
(b) hybridizing the partially double-stranded polynucleotide(s) from the first sample of biological material to one or more sets of nucleic acid probes and hybridizing the partially double-stranded polynucleotide(s) from the second sample of biological material to one or more sets of nucleic acid probes; and
(c) detecting or determining the presence or amount of partially double-stranded polynucleotide(s) from the first sample of biological material relative to the partially double-stranded polynucleotide(s) from the second sample of biological material.

22. The method according to claim 21, wherein the first sample of biological material comprises one or more cells, a cell lysate, or subcellular fraction, and the second sample of biological material also comprises one or more cells, a cell lysate, or subcellular fraction, and wherein the first and second sample differ in cell type, tissue type, physiological state, disease state, radiological, or biological treatment, or developmental stage.

23. The method according to claim 22, wherein the first sample of biological material is chosen from a cancerous cell population, and the second sample of biological material is chosen from a reference cell population of the same cell type as the cancerous cell population.

24. The process according to claim 21, wherein all of the partially double-stranded polynucleotide(s) contain at least one detectable label.

25. The method according to claim 24, wherein the detectable label is selected from a radioisotope, a chromophore, a fluorophore, an enzyme, an antigen, a reactive group, and a double-stranded DNA selective reagent.

26. The method according to claim 21, wherein one or more sets of nucleic acid probes is attached to a solid support.

27. The process according to claim 26, wherein the solid support is chosen from capillary tubes, beads, fibers, slides, sheets, pins, microtiter plates, silicon, porous silicon, porous metal oxide, plastic, polycarbonate, polystyrene, cellulose, nitrocellulose, nylon, PVDF, glass, TEFLON®, polystyrene divinyl benzene, aluminum, carbon, steel, iron, copper, nickel, silver, and gold.

28. The method to claim 21, wherein one or more sets of nucleic acid probes comprise a DNA microarray.

29. The method according to claim 21, wherein a second primer, P1, is used in the generation of at least one partially double-stranded polynucleotide.

30. The method according to claim 29, wherein P1 contains a detectable label.

31. The method according to claim 30, wherein the P1 used for amplification of the target nucleic acid from the first sample of biological material may contain the same label or a different label than the P1 used for amplification of the target nucleic acid from the second sample of biological material.

32. The method according to claim 31, wherein the label(s) is detected by FRET.

33. A partially double-stranded polynucleotide comprising three regions, wherein:
a) the first region comprises a single-stranded region of at least 8 nucleotides, wherein the single-stranded region was generated by:
(i) providing a first primer, P1, containing at least one labile nucleotide;
(ii) providing a second primer, P1, comprising a sequence that is specific for a target nucleic acid sequence and optionally contains a detectable label and/or a labile nucleotide;
(iii) amplifying the target nucleic acid sequence with P1, and P1 to generate a double-stranded amplicon containing at least one labile nucleotide;
(iv) exposing the double-stranded amplicon to conditions that promote single-strand cleavage of the amplicon at the site of the at least one labile nucleotide; and
(v) exposing the cleaved amplicon to conditions which promote the dissociation of the cleaved portions from the first region;
b) the second region comprises the sequence of the double-stranded amplicon between the first region and a third region; and
c) the third region comprises the sequence of the double-stranded amplicon comprising P1 and the complementary sequence to P1.

34. The amplicon of claim 33, wherein the single-stranded region is at least 10 nucleotides in length.

35. The amplicon of claim 33, wherein the single-stranded region is at least 20 nucleotides in length.

36. The amplicon of claim 33 containing a detectable label.

37. The amplicon of claim 33, wherein the detectable label is selected from a radioisotope, a chromophore, a fluorophore, an enzyme, an antigen, a reactive group, and a double-stranded DNA selective reagent.

38. The amplicon of claim 33 wherein the labile nucleotide is dUTP, and wherein the single-stranded cleavage of the amplicon at the site of the labile nucleotide occurs by treatment with uracil N-glycosylase.

39. A method for generating a partially double-stranded polynucleotide containing at least one single-stranded index sequence at a terminal end comprising:
a) preparing a first primer, Pa, comprising two regions: (i) a first region comprising a first index sequence containing at least one labile nucleotide wherein, the first region is not complementary to a target nucleic acid sequence and (ii) a second region comprising a sequence that is specific for the target nucleic acid sequence;
b) preparing a second primer, Pb, comprising a sequence that is specific for the target nucleic acid sequence and optionally contains a detectable label and/or a second index sequence, wherein said first and second index sequences may be the same or different;
c) amplifying the target nucleic acid sequence with Pa and Pb to generate a double-stranded amplicon from both the first sample and the second sample;
d) exposing the amplicons to conditions that promote single-stranded cleavage of the amplicons at the site of the labile nucleotide(s);
e) exposing the cleaved amplicons to conditions that promote the dissociation of the cleaved portions of the index region of the primer containing the labile nucleotide to generate a single-stranded region at the terminal end.

40. The method according to claim 39, wherein the double-stranded amplicon contains a detectable label.

41. The method according to claim 40, wherein the detectable label is selected from a radioisotope, a chromophore, a fluorophore, an enzyme, an antigen, a reactive group, and a double-stranded DNA selective reagent.

42. The method according to claim 39, wherein the labile nucleotide is dUTP, and wherein the single-stranded cleavage of the amplicon at the site of the labile nucleotide occurs by treatment with uracil N-glycosylase.

43. The method according to claim 39, wherein the target nucleic acid is chosen from mRNA, cDNA, genomic DNA, recombinant DNA, plasmid DNA, and amplified DNA.

44. The method according to claim 39, wherein part (c) involves the amplification of a single target nucleic acid sequence.

45. The method according to claim 39, wherein part (c) involves the amplification of 2-1000 target nucleic acid sequences.

46. A partially double-stranded amplicon containing at least one single-stranded index region at a terminal end prepared according to the method of claim 39.

47. A method for detecting or determining the presence or amount of a target nucleic acid sequence in a first sample of biological material relative to the same target nucleic acid sequence in a second sample of biological material comprising:
a) preparing a first primer, Pa, comprising two regions: (i) a first region comprising a first index sequence containing at least one labile nucleotide and wherein, the first region is not complementary to the target nucleic acid sequence and (ii) a second region comprising a sequence that is specific for the target nucleic acid sequence;
b) preparing a second primer, Pb, comprising a sequence that is specific for the target nucleic acid sequence and optionally contains a detectable label and/or a second index sequence, wherein said first and second index sequences may be the same or different;
c) amplifying the target nucleic acid sequence with Pa and Pb to generate a double-stranded amplicon from both the first sample and the second sample;
d) exposing the amplicons to conditions that promote single-stranded cleavage of the amplicons at the site of the labile nucleotide(s);
e) exposing the cleaved amplicons to conditions that promote the dissociation of the cleaved portions of the index region of the primer containing the labile nucleotide to generate a single-stranded region at the terminal end;
f) hybridizing the single-stranded index region(s) of the partially double-stranded amplicon from the first sample of biological material to complementary single-stranded index nucleic acid sequence(s) bound to a first solid support, and hybridizing the single-stranded index region(s) of the partially double-stranded amplicon from the second sample of biological material to complementary single-stranded index nucleic acid sequence(s) bound to a second solid support, wherein said first and second solid support may be the same or different; and
g) detecting or determining the presence or amount of the amplicon from the first sample of biological material relative to the amplicon from the second sample of biological material.

48. The method according to claim 47, wherein the solid support is chosen from capillary tubes, beads, fibers, slides, sheets, pins, microtiter plates, silicon, porous silicon, porous metal oxide, plastic, polycarbonate, polystyrene, cellulose, nitrocellulose, nylon, PVDF, glass, TEFLON®, polystyrene divinyl benzene, aluminum, carbon, steel, iron, copper, nickel, silver, and gold.

49. The method according to claim 47, wherein part (c) involves the amplification of 2 to 1000 target nucleic acid sequences.

50. The method according to claim 47, wherein the solid support comprises 2 to 1000 single-stranded index regions.
